SEQUENCE LISTING (2 sequences)

SEQ ID NO: 1 - protein, 263 aa, Sus scrofa
MWSLVMRLALSLAGTGAAPPIQSRIIGGRECEKDSHPWQVAIYHYSSFQCGGVLVDPKWVLTAAHCKNDNYQVWLGRHNLFENEVTAQFFGVTADFPHPGFNLSLLKNHTKADGKDYSHDLMLLRLQSPAKITDAVKVLELPTQEPELGSTCQASGWGSIEPGPDDFEFPDEIQCVELTLLQNTFCADAHPDKVTESMLCAGYLPGGKDTCMGDSGGPLICNGMWQGITSWGHTPCGSANKPSIYTKLIFYLDWINDTITENP

SEQ ID NO: 2 - protein, 262 aa, Homo sapiens
MWFLVLCLALSLGGTGAAPPIQSRIVGGWECEQHSQPWQAALYHFSTFQCGGILVHRQWVLTAAHCISDNYQLWLGRHNLFDDENTAQFVHVSESFPHPGFNMSLLENHTRQADEDYSHDLMLLRLTEPADTITDAVKVVELPTEEPEVGSTCLASGWGSIEPENFSFPDDLQCVDLKILPNDECKKAHVQKVTDFMLCVGHLEGGKDTCVGDSGGPLMCDGVLQGVTSWGYVPCGTPNKPSVAVRVLSYVKWIEDTIAENS

(19) United States
(12) Patent Application Publication
(10) Pub. No.: US 2012/0201804 A1
(43) Pub. Date: Aug. 9, 2012

(54) TISSUE KALLIKREIN FOR THE TREATMENT OF SCHIZOPHRENIA AND BIPOLAR DISORDER

(75) Inventors: Mark Williams, Winnipeg (CA); Matthew Charles, Winnipeg (CA)

(73) Assignee: Sanomune, Inc., Winnipeg (CA)

(21) Appl. No.: 13/278,933

(22) Filed: Oct. 21, 2011

Related U.S. Application Data
(63) Continuation of application No. PCT/CA2010/000561, filed on Apr. 21, 2010.
(60) Provisional application No. 61/171,189, filed on Apr. 21, 2009.

(51) Int. Cl. A61K 38/48; A61P 25/18

(52) U.S. Cl. 424/94.64

(57) ABSTRACT

The invention includes methods of treating psychiatric disorders including schizophrenia, associated conditions of the schizophrenic spectrum and bipolar disorder, comprising administering tissue kallikrein (KLK1), variants or active fragments thereof. The invention also includes compositions comprising KLK1, variants, or active fragments thereof.

### CROSS REFERENCE TO RELATED APPLICATIONS

This application claims the benefit of and priority to U.S. Patent Application No. 61/171,189, filed Apr. 21, 2009, under the title TISSUE KALLKREIN FOR THE TREATMENT OF SCHIZOPHRENIA AND BIPOLAR DISORDER.

The content of the above patent application is hereby expressly incorporated by reference into the detailed description hereof.

### FIELD OF THE INVENTION

The present invention provides methods of treating psychiatric disorders including schizophrenia, associated conditions of the schizophrenic spectrum and bipolar disorder, comprising administering tissue kallikrein (KLK1), variants or active fragments thereof.

### BACKGROUND OF THE INVENTION

Schizophrenia is a debilitating mental illness that affects approximately 1% of the world population at large (Lang et al., Cell Physiol Biochem., 2007, 20:687-702). This complex, chronic brain disorder displays a variety of symptoms that are classified as positive, negative, or cognitive impairment type and begin to appear in adolescence or early adulthood.

The first drugs developed to treat schizophrenia, termed typical antipsychotics, are high affinity dopamine D2 receptor antagonists which prevent excessive levels of dopamine primarily thought to be responsible for the psychotic positive symptoms of the disease. Ultimately, this group of drugs fell out of favor because of their common undesirable side effects (sleepiness, drowsiness, and extrapyramidal symptoms like tardive dyskinesia), that lead to non-compliance and increased likelihood of psychotic relapse (Lieberman et al., Am J Psychiatry, 2003, 160:1396-1404); (Asher-Svanum et al., BMC Psychiatry, 2006, 6:8); (Lieberman et al., N Engl J. Med., 2005, 353:1209-23).

Atypical antipsychotics were designed to have reduced side effects by having lower affinity to the dopamine D2 receptor or higher affinity serotonin receptor targeting. This group of drugs exhibits reduced extrapyramidal symptom side effects, but is hampered by other side effects, including increased weight gain and onset of diabetes (Leiberman et al., 2005). In addition, atypical antipsychotics are unable to adequately alleviate negative and cognitive impairment symptoms (Leiberman et al., 2005).

Another treatment for schizophrenia is the use of mood-stabilizers such as lithium. Mood-stabilizers treat the mania (a positive symptom) and depression (a negative symptom) of the disease. However, lithium must be used at a dose that is almost toxic to the body. As such, constant monitoring is required to prevent kidney toxicity, dehydration, convulsions, and tremors that can be fatal. In addition, lithium also has common undesirable side effects, such as numbness, dazed feeling, and drowsiness. An extensive review has shown that lithium treatment alone has no effect for schizophrenia (Leucht et al., Cochrane Database Syst Rev., 2007 3:CD001258).

Bipolar disorder (BP) is believed to affect 2.6% of the population in the United States (Muzina et al., Ann Clin Psychiatry 2007, 19(4):305-12). However, the actual number of individuals affected by this disorder may be greater due to inconsistent diagnosis. Bipolar disorder is characterized by periods of depression and periods of mania which both greatly disrupt everyday life. Bipolar disorder can be classified in two groups (either bipolar I disorder or bipolar II disorder), and both of these groups include symptoms of depression and mania. Mania can be defined as racing thoughts, rapid speech, elevated levels of activity and agitation as well as an inflated sense of self-esteem while the depression phase includes feeling a lack of self worth, isolation, sadness, feeling overwhelmed and may include suicidal thoughts. This debilitating condition requires medical intervention, often including the use of pharmaceuticals for regulation of the symptoms.

Typical treatments include mood stabilizers, antimanic agents, anticonvulsants and antiepileptics which can include lithium, chlorpromazine, aminophenylpyridone, aripiprazole, olanzapine and fluoxetine, just to name a few. As previously mentioned these drugs can have several undesirable side effects. In particular lithium is known to cause nausea, vomiting, tremor and diarrhea. Hospitalization is often required during the treatment of manic periods (Muzina Prim Care 2007, 34(3):521-50).

The current treatments available for schizophrenia and bipolar disorder are far from satisfactory. Issues of undesirable side effects and inadequate alleviation of symptoms still persist and thus, better therapies are required (Javitt et al., Biol Psychiatry. 1999, 45:668-679). Further study into schizophrenia has revealed additional mechanisms underlying its cause. Improper glutamate signaling is a key factor rather than dopamine, which is now thought to be a consequence of improper glutamate signaling (Buchanan et al., Schizophr Bull., 2007, 33:1120-1130); (Depoortere et al., Neuropsychopharmacology, 2005, 30:1963-1985).

The use of phencyclidine (PCP), an N-methyl D-aspartate (NMDA) receptor (NMDAR) antagonist, in animal models reproduces all symptoms (positive, negative and cognitive impairment) of schizophrenia by preventing glutamate from binding to the NR2 portion of the NMDA receptor in brain cells (Javitt et al., 1999). Kynurenic acid (a naturally occurring NMDA receptor antagonist) is found at abnormally high levels in those with schizophrenia and is linked to memory problems (Chess et al., Schizophr Bull., 2007, 33:797-804). Furthermore, up to 80% of those with Systemic Lupus Erythematosus suffer psychosis and cognitive impairments similar to schizophrenia due to anti-DNA antibodies that cross react with the NR2 portion of the NMDA receptor in the brain. This phenotype can be replicated in animal models by transferring human SLE anti-DNA antibodies into the brain (DeGiorgio et al., Nat. Med., 2001, 7:1189-93)

Molecular analysis of NMDA receptor antagonism has revealed decreased phosphorylation of Akt and GSK-3β in the PI3K/Akt/GSK-3β pathway (Lei et al., Neuropsychopharmacology, 2007, doi: 10.1038/sj.npp.1301511). Schizophrenia patients exhibit decreased Akt activity compared to normal individuals (Emamian et al., Nat. Med., 2004, 36:131-137), such that its inhibitory regulation of GSK-3β is likely impaired. GSK-3β is a serine-threonine kinase implicated in neuronal degeneration and apoptosis, which may lead to the loss of neuronal synaptic connectivity and volume. In schizophrenia, this contributes to associated negative and cognitive impairment symptoms (Benitez-King et al., Curr. Drug Targets CNS Neurol Disord., 2004, 3:515-33). Interestingly, psychoactive drugs that induce temporary hallucinogenic positive-like symptoms disrupt the neuronal cytoskeleton (degeneration)(Benitez-King et al., 2004), while over expression of β-catenin, a transcription factor inhibited by GSK-3β activity, leads to mood stabilization similar to lithium treatment (Gould et al., Neuropsychopharmacology, 2007, 32:2173-83).

Rowe et al. demonstrates that lithium modulates GSK-3 when administered to treat bipolar disorder (Rowe et al., Neurosci Biobehav Rev. 2007, 31(6):920-31). This suggests that lithium is effective for treating bipolar disorder as well as other conditions caused by dysfunction of the GSK (P13K/Akt/GSK-3β) regulatory pathway.

Additional molecular analysis has shown that levels of neurotrophins, such as nerve growth factor (NGF), are decreased in schizophrenia patients. Mature neurotrophins usually activate the PI3K/Akt/GSK-3β pathway by binding to their appropriate Trk receptor, which also leads to their increased expression. Neurotrophins are noted for their ability to provide neuroprotection, induce axonal growth and associated synaptic connectivity, and neurogenesis.

Treatment strategies disclosed herein will address a variety of symptoms of schizophrenia and bipolar disorder that are not found in current treatments for the disease.

### BRIEF DESCRIPTION OF THE FIGURES

FIGS. 1-3 show schematic forms of the biochemical pathways thought to be implicated in schizophrenia and bipolar disorder, and KLK1's possible route of action.

### SUMMARY OF THE INVENTION

The present invention includes methods of treating a psychiatric disorder that are affected by the P13K/Akt/GSK-3β pathway comprising administering tissue kallikrein (KLK1), variants or active fragments thereof. A psychiatric disorder can be schizophrenia, associated conditions of the schizophrenic spectrum, or bipolar disorder.

One aspect of the invention provides a method to treat symptoms of schizophrenia and associated conditions comprising administering tissue kallikrein, variants or active fragments thereof. In a further aspect, a symptom of schizophrenia can be a positive symptom, a negative symptom, or a cognitive symptom. Positive symptoms can include, but are not limited to, delusions, hallucinations, and catatonic behavior. Negative symptoms can include, but are not limited to, lack of emotion, inability to enjoy activities, low energy, lack of interest in life, alogia, inappropriate social skills, inability to make friends, and social isolation. Cognitive symptoms include, but are not limited to, impairment of attention/information processing, sensory gating, problem solving, processing speed, verbal and visual learning and memory, and working memory.

In a further aspect, the invention includes a method of treating the prodomal stage of first onset or relapse of schizophrenia, or bipolar disorder comprising administering KLK1, or a variant or an active fragment thereof.

An embodiment of the invention includes treating a symptom of bipolar disorder comprising administering KLK1, or a variant or an active fragment thereof. A symptom of bipolar disorder can be continuous mood disruption, which includes both periods of depression and mania.

In a further aspect, the invention includes a method of increasing neuroprotection of brain cells by administering KLK1, or a variant or an active fragment thereof. Neuroprotection can include, but is not limited to, an increase of neurotrophins (e.g., NGF) and/or a decrease in GSK-3β activity.

In a further aspect, the invention includes a method of preventing apoptosis of cells in the brain by administering KLK1, or a variant or an active fragment thereof. A method of preventing apoptosis includes, but is but not limited to, increasing neurotrophins such as NGF, and/or decreasing GSK-3β activity.

In a further aspect, the invention provides a method of preventing neurodegeneration in the brain by administering KLK1, or a variant or an active fragment thereof. A method of preventing neurodegeneration includes, but is not limited to, increasing neurotrophins such as NGF, and/or a decrease in GSK-3 β activity.

In another aspect of the present invention, KLK1, or a variant or an active fragment thereof, can be administered orally. Oral administration may be an enteral administration. Oral formulations can be liquids, pills, solution, tablets, sustained release capsules, enteric coated capsules, or syrups. An oral therapeutic dose can be a maximum dose range of about 1 to about 1000 International Units (IU) per day.

In another aspect of the present invention, KLK1, or a variant or an active fragment thereof, can be administered intranasally. Formulations for intranasal administration can be ointments, creams, lotions, pastes, gels, sprays, aerosols, oils, and the like. A nasal therapeutic dose is a maximum dose of about 1 to about 5000 IU per day.

Another aspect of the present invention includes treatment and prevention methods as described herein, further comprising concurrent administration of a therapeutic compound useful for treating schizophrenia or bipolar disorder. Therapeutic compounds useful for treating schizophrenia and/or bipolar disorder include, but are not limited to, typical and atypical antipsychotics, and mood stabilizers such as lithium and valproic acid.

Another aspect of the present invention includes a pharmaceutical composition formulated for oral administration comprising about 1 to about 1000 IU of KLK1, or a variant or an active fragment thereof, optionally further comprising a pharmaceutically acceptable excipient, and optionally further comprising an additional therapeutic compound as described above.

Another aspect of the present invention includes a pharmaceutical composition formulated for intranasal administration comprising about 1 to about 5000 IU of KLK1, or a variant or an active fragment thereof, optionally comprising a pharmaceutically acceptable excipient.

### DETAILED DESCRIPTION

### Definitions

“Tissue kallikrein” or “KLK1” is a serine protease that is primarily noted for its role in controlling hypertension through its cleavage of kininogen into lysyl-bradykinin (kallidin) (Yousef et al., Endocrine Rev., 2001; 22: 184-204). There are a large number of enzymes in the KLK family, but KLK1 appears to be a ubiquitous or multiple target acting enzyme. As used herein, the term “tissue kallikrein” is synonymous with the following terms: callicrein, glumorin, padreatin, padutin, kallidinogenase, bradykininogenase, pancreatic kallikrein, onokrein P, dilminal D, depot-Padutin, urokallikrein, or urinary kallikrein.

Tissue kallikrein polypeptide has the following sequence:

NP_001001911 GI: 50054435 Sus scrofa
1-17 signal peptide
18-24 propeptide
25-263 mature peptide
>gi|50054435|ref|NP_001001911.1| kallikrein 1 [Sus scrofa]
(SEQ ID NO: 1)
MWSLVMRLALSLAGTGAAPPIQSRIIGGRECEKDSHPWQVAIYHYSSFQCGGVLVDPKWVLTAAHCKNDN
YQVWLGRHNLFENEVTAQFFGVTADFPHPGFNLSLLKNHTKADGKDYSHDLMLLRLQSPAKITDAVKVLE
LPTQEPELGSTCQASGWGSIEPGPDDFEFPDEIQCVELTLLQNTECADAHPDKVTESMLCAGYLPGGKDT
CMGDSGGPLICNGMWQGITSWGHTPCGSANKPSIYTKLIFYLDWINDTITENP

Another embodiment includes:

NP_002248 GI: 4504875 Homo sapiens
1-18 signal peptide
19-24 propeptide
25-262 mature peptide
>gi|4504875|ref|NP_002248.1| kallikrein 1 preproprotein [Homo sapiens]
(SEQ ID NO: 2)
MWFLVLCLALSLGGTGAAPPIQSRIVGGWECEQHSQPWQAALYHFSTFQCGGILVHRQWVLTAAHCISDN
YQLWLGRHNLFDDENTAQFVHVSESFPHPGFNMSLLENHTRQADEDYSHDLMLLRLTEPADTITDAVKVV
ELPTEEPEVGSTCLASGWGSIEPENFSFPDDLQCVDLKILPNDECKKAHVQKVTDFMLCVGHLEGGKDTC
VGDSGGPLMCDGVLQGVTSWGYVPCGTPNKPSVAVRVLSYVKWIEDTIAENS

The term “active fragment” refers to smaller portions of the KLK1 polypeptide that retain the activity of the full-length KLK1 polypeptide.

A “variant” or “mutant” of a starting or reference polypeptide is a polypeptide that 1) has an amino acid sequence different from that of the starting or reference polypeptide and 2) was derived from the starting or reference polypeptide through either natural or artificial (manmade) mutagenesis. Such variants can include an amino acid deletion, insertion, substituting, or combinations thereof. A variant amino acid, in this context, refers to an amino acid different from the amino acid at the corresponding position in a starting or reference polypeptide sequence (such as that of a source antibody or antigen binding fragment). Any combination of deletion, insertion, and substitution may be made to arrive at the final variant or mutant construct, provided that the final construct possesses the desired functional characteristics. The amino acid changes also may alter post-translational processes of the polypeptide, such as changing the number or position of glycosylation sites. Methods for generating amino acid sequence variants of polypeptides are described in U.S. Pat. No. 5,534,615, expressly incorporated herein by reference.

A “wild type” or “reference” sequence or the sequence of a “wild type” or “reference” protein/polypeptide maybe the reference sequence from which variant polypeptides are derived through the introduction of mutations. In general, a “wild type” sequence for a given protein is a sequence that is most common in nature. Similarly, a “wild type” gene sequence is the sequence for that gene which is most commonly found in nature. Mutations may be introduced into a “wild type” gene (and thus the protein it encodes) either through natural processes or through man induced means. The products of such processes are “variant” or “mutant” forms of the original “wild type” protein or gene.

“Percent (%) amino acid sequence identity” with respect to the polypeptides identified herein is defined as the percentage of amino acid residues in a candidate sequence that are identical with the amino acid residues in the reference sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. The ALIGN-2 program is publicly available through Genentech, Inc., South San Francisco, Calif.

For purposes herein, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y,

where X is the number of amino acid residues scored as identical matches by the sequence alignment program in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

“Percent (%) nucleic acid sequence identity” is defined as the percentage of nucleotides in a candidate sequence that are identical with the nucleotides in a reference polypeptide-encoding nucleic acid sequence, after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For purposes herein, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) is calculated as follows:

100 times the fraction W/Z,

where W is the number of nucleotides scored as identical matches by the sequence alignment program in that program's alignment of C and D, and where Z is the total number of nucleotides in D. It will be appreciated that where the length of nucleic acid sequence C is not equal to the length of nucleic acid sequence D, the % nucleic acid sequence identity of C to D will not equal the % nucleic acid sequence identity of D to C. It is fairly common in the art that “homologous sequences”, for example, sequences with 80% identity to a known sequence, have very similar activity to the known sequence.

The term “amino acid” is used in its broadest sense and is meant to include the naturally occurring L α-amino acids or residues. The commonly used one and three letter abbreviations for naturally occurring amino acids are used herein (Lehninger, A. L., Biochemistry, 2d ed., pp. 71-92, (1975), Worth Publishers, New York). The term includes all D-amino acids as well as chemically modified amino acids such as amino acid analogs, naturally occurring amino acids that are not usually incorporated into proteins such as Norleucine, and chemically synthesized compounds having properties known in the art to be characteristic of an amino acid. For example, analogs or mimetics of phenylalanine or proline, which allow the same conformational restriction of the peptide compounds as natural Phe or Pro are included within the definition of amino acid. Such analogs and mimetics are referred to herein as “functional equivalents” of an amino acid. Other examples of amino acids are listed by Roberts and Vellaccio, In: The Peptides: Analysis, Synthesis, Biology, Gross and Meiehofer, Eds., Vol. 5 p 341, Academic Press, Inc, N.Y. 1983, which is incorporated herein by reference.

The term “protein” has an amino acid sequence that is longer than a peptide. A “peptide” contains 2 to about 50 amino acid residues. The term “polypeptide” includes proteins and peptides. Examples of proteins include, but are not limited to, antibodies, enzymes, lectins and receptors; lipoproteins and lipopolypeptides; and glycoproteins and glycopolypeptides.

A “fusion protein” and a “fusion polypeptide” refer to a polypeptide having two portions covalently linked together, where each of the portions is a polypeptide having a different property. A property may be a biological property, such as activity in vitro or in vivo. A property may also be a simple chemical or physical property, such as binding to a target antigen, catalysis of a reaction, etc. The two portions may be linked directly by a single peptide bond or through a peptide linker containing one or more amino acid residues. Generally, the two portions and the linker will be in reading frame with each other. Preferably, the two portions of the fusion polypeptide are obtained from heterologous or different polypeptides.

The term “therapeutically effective amount” refers to an amount of a composition effective to “alleviate” or “treat” a disease or disorder in a subject or mammal. Generally, alleviation or treatment of a disease or disorder involves the lessening of one or more symptoms or medical problems associated with the disease or disorder. In an embodiment, a “therapeutically effective amount” is an amount that treats schizophrenia and the associated symptoms of schizophrenia, the prodomal stage of first onset, relapse of schizophrenia, associated conditions of schizophrenic spectrum and bipolar disorder.

The terms “treatment” and “treating” refer to inhibiting, alleviating, and healing a disease and conditions or symptoms thereof. “Treating” or “treatment” refers to both therapeutic treatment and prophylactic or preventative measures, wherein the object is to prevent or slow down (lessen) the targeted pathologic condition or disorder. Treatment includes administering KLK1, or a variant or an active fragment thereof to a patient with schizophrenia or bipolar disorder. Administration of KLK1, or a variant or active fragment thereof, includes a “therapeutically effective amount” which includes a prophylactic amount (e.g., an amount effective for alleviating or healing the above mentioned diseases or symptoms thereof). Successful treatment and improvement of the disease can be assessed by psychiatric evaluation.

The terms “prevention” and “prevent” refers prophylaxis, i.e., to keep a disease, disease condition, disease pathology, and/or disease symptoms from happening. Schizophrenia and bipolar disease can be prevented by administering a therapeutically effective amount of KLK1, or a variant or an active fragment thereof. A “therapeutically effective amount” as used herein includes a prophylactic amount (e.g., an amount effective for preventing the above mentioned diseases or symptoms thereof). Successful treatment and improvement of the disease can be assessed by psychiatric evaluation.

The term “schizophrenia” refers to a chronic psychological/mental disorder which can be characterized by psychosis (loss of contact with reality), hallucinations (false perceptions), delusions (false beliefs), disorganized speech and behavior, flattened effect (restricted range of emotions), cognitive deficits (impaired reasoning and problem solving) and occupational and social dysfunction. Symptoms of schizophrenia may be classified by a skilled physician based on medical history, interview consultation, physical examination and lab tests. The term “schizophrenia” for use herein encompasses all subtypes of schizophrenia, including, but not limited to disorganized type, catatonic type, paranoid type, residual type and undifferentiated type (The Merck Manual of Diagnosis and Therapy, 2006)

Disorders related to schizophrenia include: a) brief psychotic disorder, b) delusional disorder and c) schizoaffective disorder.

The term “bipolar disorder” or “BP” or “manic depressive disorder” or “manic depressive illness” refers to a chronic psychological/mood disorder which can be characterized by significant mood changes including periods of depression and euphoric manic periods. BP is diagnosed by a skilled physician based on personal and medical history, interview consultation and physical examinations.

The term “mania” or “manic periods” refers to periods where an individual exhibits some or all of the following characteristics: racing thoughts, rapid speech, elevated levels of activity and agitation as well as an inflated sense of self-esteem, euphoria, poor judgment, insomnia, impaired concentration and aggression.

The term “depression” refers to periods where an individual with a depressed mood meaning a mental state that produces feelings including but not limited to sadness and discouragement, which may result in destructive behaviors.

The term “prodomal stage of first onset” refers to early symptoms and signs of an illness that occur before characteristic manifestations are seen in a fully developed illness. In schizophrenia and bipolar disorder, this represents a period of prepsychotic disturbance or an interval between onset of usual behaviour disturbance and first signs of prominent psychotic symptoms. A prodomal period can be before the first behavioural episode of a schizophrenic or individual with bipolar disorder or a period before the relapse of the episode.

The term “relapse of schizophrenia” refers to regression of symptoms of a healthy individual formerly affected with schizophrenia back to a state at which the were upon schizophrenia affection as diagnosed by a psychiatrist.

The term “relapse of bipolar disorder” refers to regression of symptoms of a healthy individual formerly affected with bipolar disorder back to a state at which they were upon bipolar disorder affection as diagnosed by a psychiatrist.

The term “increase neuroprotection” or “increased neuroprotection” refers to inhibition of neuronal damage and death.

The terms “concurrent administration”, “concurrently administering” and “administered concurrently” refers to administering KLK1 and a therapeutic compound in admixture. For example, a pharmaceutical composition can be separate compounds or separate pharmaceutical compositions administered consecutively, simultaneously, or at different times. Preferably, KLK1 and a therapeutic compound are administered simultaneously.

The term “additional therapeutic compound” refers to a therapeutic used for treating schizophrenia or bipolar disorder other than KLK1, a variant or a fragment thereof. An additional therapeutic compound for treating schizophrenia or bipolar disorder includes, but is not limited to, typical and atypical antipsychotics and mood stabilizers such as lithium and valproic acid.

The term “schizophrenia spectrum” refers to closely related psychotic conditions that share a variety of symptoms in common yet differ in severity ranging from mild to severe. Schizoid personality disorder is located in the mild range of the spectrum, schizotypal personality falls in the middle, and schizophrenia is at the severe end of the spectrum. Common symptoms and cognitive impairments found across the whole spectrum are due to shared genetic and environmental events which lead to abnormal temporal brain structures in comparison to normal individuals. The more severe end of the spectrum, chronic schizophrenia, displays additional abnormalities in brain structure like the frontal lobe deficits accounting for worse symptoms while the milder end of the spectrum, schizoid personality disorder, have no such additional abnormalities and can perhaps better compensate for the temporal deficit thus milder symptoms.

The term “positive symptom” refers to, but is not limited to, delusions, hallucinations, disorganized thinking, and catatonic motor behaviors.

The term “negative symptom” refers to, but is not limited to, lack of emotion, inability to enjoy activities, low energy, lack of interest in life, alogia, avolition, inappropriate social skills, inability to make friends and social isolation.

The term “cognitive symptom” refers to, but is not limited to, abilities of attention/information processing, sensory gating, problem solving, processing speed, verbal and visual learning, and memory and working memory.

### Methods of Treating Schizophrenia and Bipolar Disorder

The present invention provides methods for treating schizophrenia, associated conditions of the schizophrenic spectrum, and bipolar disorder. One embodiment includes a method of treating schizophrenia, associated conditions of the schizophrenic spectrum, or bipolar disorder in a mammal by administering tissue kallikrein, a variant or active fragment thereof to the mammal. Administration can be oral or intranasal.

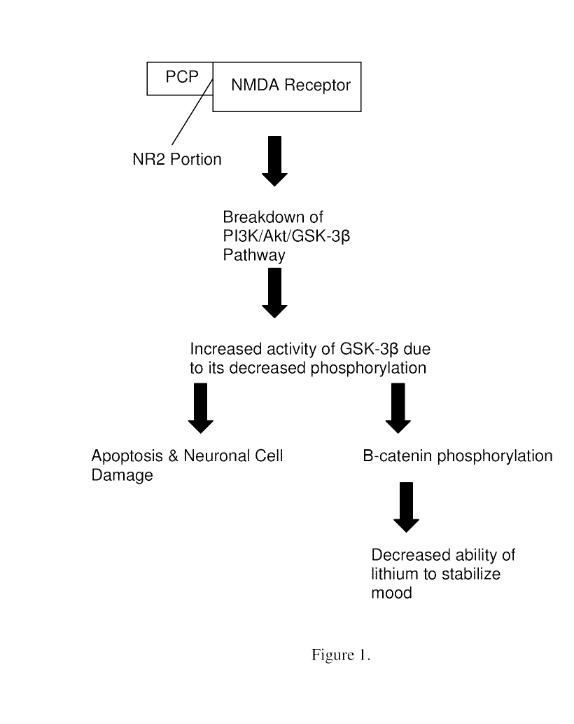

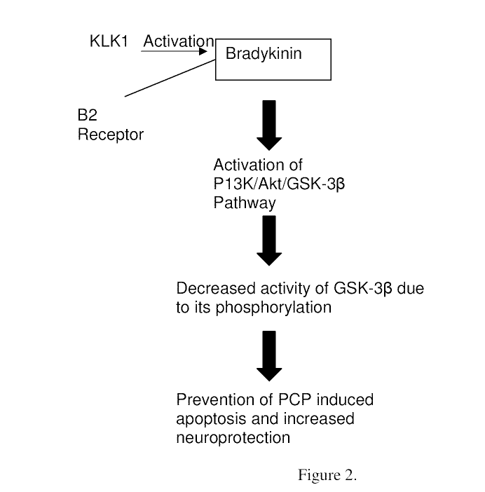

The pathology of schizophrenia, associated conditions of the schizophrenic spectrum, and bipolar disorder includes apoptosis of brian cells and other neural tissues. Administration of KLK1 provides neuroprotection, i.e., KLK1 inhibits apoptosis of brain cells. KLK1 activates the bradykinin B2 receptor, leading to activation of the PI3K/Akt/GSK-3β pathway. This activation of Akt prevents apoptosis and, thereby producing a neuroprotective effect. Activation of bradykinin B2 receptor by KLK1 also activates the regulatory inhibition of GSK-3β. This process is represented diagrammatically in FIG. 2. Additionally, inhibition of GSK-3β prevents/inhibits β-catenin phosphorylation, which then prevents/inhibits ubigitin-dependent breakdown of β-catenin. Activity of β-catenin can improve mood-stabilization ability achieved by the administration of lithium. This process is represented diagrammatically in FIG. 1. Administering KLK1, a variant or active fragment thereof, concurrently with lithium further improves mood stabilization.

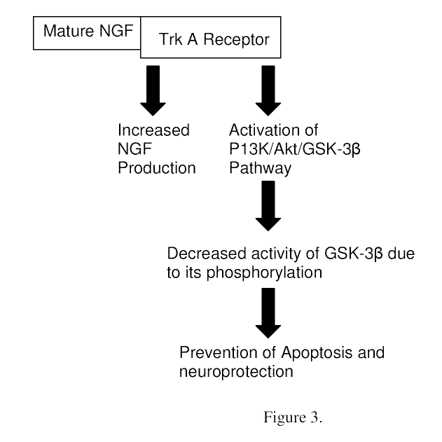

KLK1, or a variant or active fragment thereof, can also interact with neurotrophins to produce a neuroprotection from apoptosis and neurodegeneration, promote axonal growth and improve neuronal synaptic connectivity. Administering KLK1, or a variant or active fragment thereof, can modify neurotrophins, for example NGF, to provide a neuroprotective effect and thereby treat schizophrenia, conditions of the schizophrenia spectrum, and bipolar disease. The precursor form of NGF is post-transitionally modified by KLK1 cleavage into its mature form such that NGF is then able to activate the PI3K/Akt/GSK-3β pathway through Trk A. This process is represented diagrammatically in FIG. 3. The binding of NGF to Trk A, leading to Akt activation, can only take place once NGF has been cleaved into its mature form. Administration of KLK1, or a variant or active fragment thereof, also treats schizophrenia, conditions of the schizophrenia spectrum, and bipolar disease through KLK1 modifications of neurotrophins.

### Pharmaceutical Compositions

Pharmaceutical compositions of the invention include formulations to be administered orally or intranasally. Formulations suitable for intranasal administration include powder, granules, solution, drops, ointments, creams, lotions, pastes, gels, sprays, aerosols, oils and the like. Solutions or suspensions of the invention can be applied directly to the nasal cavity by conventional means, for example, with a dropper, pipette or spray. Formulations may be provided in a single or multidose form. A solution may be sterile, isotonic or hypotonic, and otherwise suitable for administration by injection or other means and may contain appropriate adjuvants, buffers, preservatives and salts. Solutions such as nose drops may contain antioxidants, buffers, and the like. Powder or granular forms of a pharmaceutical composition can be combined with a solution and with diluting, dispersing and/or surface active agents.

Formulations for aerosol administration include formulations designed for intranasal administration. An active ingredient can be provided in a pressurized pack with a suitable propellant such as a chlorofluorocarbon (CFC) (e.g., dichlorodifluoromethane, trichlorofluoromethane, or dichlorotetrafluoroethane), carbon dioxide, or other suitable gas. An aerosol may also contain a surfactant such as lecithin. A dose of drug may be controlled by a metered valve. Alternatively active ingredients may be provided in a form of a dry powder, for example a powder mix of the compound in a suitable powder base such as lactose, starch, starch derivatives such as hydroxypropylmethyl cellulose and polyvinylpyrrolidine (PVP). The powder carrier will form a gel in the nasal cavity. A powder composition may be presented in unit dose form for example in capsules or cartridges of e.g., gelatine or blister packs from which the powder may be administered by means of an inhaler.

A pharmaceutical composition formulated for intranasal administration comprises about 1 to about 5000 IU of KLK1, or a variant, or an active fragment thereof, optionally, further comprising a pharmaceutically acceptable excipient. An intranasal dose of KLK1, variant, or active fragment thereof, can be a dose of about 1 to about 5000 IU per day; about 1 to about 4000 IU per day; about 1 to about 3000 IU per day; about 1 to about 2500 IU per day; about 1 to about 2000 IU per day; about 1 to about 1000 IU per day; about 1 to about 750 IU per day; about 1 to about 500 IU per day; about 1 to about 400 IU per day; about 1 to about 300 IU per day; about 1 to about 250 IU per day; about 1 to about 200 IU per day; about 1 to about 150 IU per day; about 1 to about 100 IU per day; about 1 to about 75 IU per day; about 1 to about 50 IU per day; about 1 to about 50 IU per day; about 1 to about 25 IU per day; about 1 to about 20 IU per day; about 1 to about 15 IU per day; about 1 to about 10 IU per day; about 1 to about 5 IU per day; about 5 to about 1000 IU per day; about 10 to about 1000 IU per day; about 15 to about 1000 IU per day; about 20 to about 1000 IU per day; about 25 to about 1000 IU per day; about 50 to about 1000 IU per day; about 75 to about 1000 IU per day; about 100 to about 1000 IU per day; about 150 to about 1000 IU per day; about 200 to about 1000 IU per day; about 250 to about 1000 IU per day; about 300 to about 1000 IU per day; about 400 to about 1000 IU per day; about 500 to about 1000 IU per day; about 750 to about 1000 IU per day; about 10 to about 100 IU per day; about 10 to about 250 IU per day; about 10 to about 500 IU per day; about 50 to about 250 IU per day; about 50 to about 500 IU per day; about 100 to about 250 IU per day; about 100 to about 500 IU per day; or about 250 to about 750 IU per day.

Formulations suitable for oral administration include liquids, pills, solution, tablets, sustained release capsules, enteric coated capsules or syrups. A pharmaceutical composition formulated for oral administration comprises about 1 to 1000 IU of KLK1, or a variant or an active fragment thereof, optionally further comprising a pharmaceutically acceptable excipient. An oral dose of KLKI, variant, or active fragment thereof, can be a dose of about 1 to about 1000 IU per day; about 1 to about 750 IU per day; about 1 to about 500 IU per day; about 1 to about 400 IU per day; about 1 to about 300 IU per day; about 1 to about 250 IU per day; about 1 to about 200 IU per day; about 1 to about 150 IU per day; about 1 to about 100 IU per day; about 1 to about 75 IU per day; about 1 to about 50 IU per day; about 1 to about 50 IU per day; about 1 to about 25 IU per day; about 1 to about 20 IU per day; about 1 to about 15 IU per day; about 1 to about 10 IU per day; about 1 to about 5 IU per day; about 5 to about 1000 IU per day; about 10 to about 1000 IU per day; about 15 to about 1000 IU per day; about 20 to about 1000 IU per day; about 25 to about 1000 IU per day; about 50 to about 1000 IU per day; about 75 to about 1000 IU per day; about 100 to about 1000 IU per day; about 150 to about 1000 IU per day; about 200 to about 1000 IU per day; about 250 to about 1000 IU per day; about 300 to about 1000 IU per day; about 400 to about 1000 IU per day; about 500 to about 1000 IU per day; about 750 to about 1000 IU per day; about 10 to about 100 IU per day; about 10 to about 250 IU per day; about 10 to about 500 IU per day; about 50 to about 250 IU per day; about 50 to about 500 IU per day; about 100 to about 250 IU per day; about 100 to about 500 IU per day; or about 250 to about 750 IU per day.

### Administration of the Pharmaceutical Composition

Traditional modes of drug administration to treat aliments in the brain include oral as well as intravenous routes of administration. These modes are not always ideal. Oral administration of compounds results in limited bioavailability (solubility, 1^{st} pass liver degradation, blood brain barrier restriction) as well as time release issues with potentially undesirable gastrointestinal side effects. However, tissue kallikrein (KLK1) appears able to pass through and may bypass the blood-brain-barrier such that it produces its effects on the brain.

Intravenous (i.v.) administration may require trained medical professionals, which is time consuming, costly to the health care system, and may result in patient compliance issues. Risks associated with intravenous administration are also present (e.g., infection at the injection site).

Intranasal administration allows a medicament to be ‘fast acting’ since it reaches the brain by a more direct route. Intranasal administration is convenient and virtually eliminates issues of patient compliance. Mucosal and submucosal epithelial cells are selectively permeable. Thus, proteins such as KLK1 pass through and bypass the blood-brain-barrier via an intranasal route. Intranasally administered KLK1 can produce its effects directly on the brain, thereby minimizing peripheral effects due to involvement of the olfactory region in the upper portion of the nasal pathway.

A substance administered intranasally may follow two possible routes—intraneuronal or extraneuronal. Uptake of peptides into olfactory neurons where the peptides travel along axons to bypass the blood-brain-barrier is an intraneuronal route. Passage through unique intercellular clefts in epithelia of the olfactory region is an extraneuronal route that allows peptides to diffuse into the subarachnoid space. An extraneuronal route is more preferable due to rapid passage time to the brain, avoidance of proteolytic degradation involved in intraneuronal pathways (Born et al., Nat. Neurosci., 2002, 5(6):514-6), and rapid eliciting of biological effects at multiple sites of the brain (Throne et al., Neuroscience, 2004, 127(2):481-96).

Although oral delivery is possible, a preferred route of administration is intranasal due to more direct delivery of KLK1 to desired sites of action in the brain.

Pharmaceutical compositions may be administered orally or intranasally. Formulations suitable for intranasal administration include ointments, creams, lotions, pastes, gels, sprays, aerosols, oils and the like. Solutions or suspensions are applied directly to the nasal cavity by conventional means, for example, with a dropper, pipette or spray. Formulations may be provided in a single or multidose form. For a dropper or pipette, a patient can administer an appropriate, predetermined volume of a solution or suspension. A spray can be administered by metering atomizing spray pump.

Formulations for aerosol administration, particularly to the respiratory tract, include intranasal administration. An active ingredient is provided in a pressurized pack with a suitable propellant such as a chlorofluorocarbon (CFC), for example, dichlorodifluoromethane, trichlorofluoromethane, or dichlorotetrafluoroethane, or carbon dioxide or other suitable gas. An aerosol may also contain a surfactant such as lecithin. A dose of drug may be controlled by a metered valve. Alternatively active ingredients may be provided in a form of a dry powder, for example a powder mix of the compound in a suitable powder base such as lactose, starch, starch derivatives such as hydroxypropylmethyl cellulose and polyvinylpyrrolidine (PVP). A powder carrier can form a gel in the nasal cavity. A powder composition may be presented in a unit dose form, for example in capsules, cartridges of gelatine, or blister packs. A powder can be administered by means of an inhaler.

A method of the invention includes delivering compounds to affected areas of the brain through transneuronal retrograde and anterograde transport mechanisms. Delivery of neurologic agents to the brain by that transport system may be achieved in several ways. One technique comprises delivering KLK1 alone to the nasal cavity. In this instance, chemical characteristics of KLK1 can facilitate its transport to diseased neurons in the brain. Auxiliary substances are capable of delivering KLK1 to peripheral sensory neurons and/or along neural pathways to malfunctioning areas of the brain. Peripheral nerve cells of the olfactory neural pathway can be utilized in order to deliver KLK1 to damaged neurons in those regions of the brain that are connected to the olfactory bulb.

A method of the invention delivers KLK1 to the nasal cavity of a mammal. It is preferred that KLK1 be delivered to the olfactory area in the upper third of the nasal cavity and particularly to the olfactory epithelium in order to promote transport of the agent into the peripheral olfactory neurons rather than the capillaries within the respiratory epithelium. Transport of KLK1 to the brain by means of the nervous system instead of the circulatory system so that KLK1 can be delivered to damaged neurons in the brain.

To deliver KLK1 to olfactory neurons, KLK1 alone or in combination with other substances as a pharmaceutical composition may be administered to the olfactory area located in the upper third of the nasal cavity. The composition may be dispensed intranasally as a powdered or liquid nasal spray, nose drops, a gel or ointment, through a tube or catheter, by syringe, by packtail, by pledget, or by submucosal infusion.

Oral administration includes enteral administration of solution, tablets, sustained release capsules, enteric coated capsules, and syrups.

KLK1 can be combined with a carrier and/or other adjuvants to form a pharmaceutical composition. Potential adjuvants include, but are not limited to, GM-1, phosphatidylserine (PS), and emulsifiers such as polysorbate 80. Further supplementary substances include, but are not limited to, lipophilic substances such as gangliosides and phosphatidylserine (PS).

KLK1 can be administered to the nasal cavity alone or in combination with a second therapeutic compound useful in treating schizophrenia or bipolar disorder. A second therapeutic compound useful for treating schizophrenia includes, but is not limited to trifluoperazine, fluanxol, loxapac, loxitane, etrafon, trilafon, thorazine, halopoidol, fluphenazine decanoate, aripiprazole, clozapine, ziprasidone, resperidone, questiapire, olanzapine, iloperidone (Titan/Novartis), DTA 201A (Knoll), DV 127090 (Solvayl Lundbeck), ORG 5222 (Organon), Osanetant (Sanofi-Synthelabo), and MEM 3454 (Memoray Pharmaceuticals Corp.). A second therapeutic compound useful for treating bipolar disorder includes, but is not limited to aripiprazole, asenapine (investigational), carbamazepine, fluoxetine and alanzapine, lamotrigine, lithium, olanzapine, oxcarbazepine, quetiapine, risperidone, topirimate, valproic acid, valproic acid, divalproex sodium, and ziprasidone.

In one embodiment of the invention, KLK1 can be combined with micelles comprising of lipophilic substances. Micelles may modify the permeability of the nasal membrane and enhance absorption of the agent. Lipophilic micelles can include gangliosides, particularly GM-1 ganglioside, and phosphatidylserine (PS).

Once KLK1 has crossed the nasal epithelium, KLK1 is transported along the olfactory neural pathway. KLK1 may be capable of movement within the olfactory system. In particular, neurotrophic and neuritogenic substances have demonstrated ready incorporation into nerve cell membranes and an affinity for nerve cell receptor sites.

### EXAMPLES

### Example 1

### Prevention of Pre-Pulse Inhibition of Startle Deficit after Treatment with KLK1 in Rats

In normal rats, the preceding pre-pulse attenuates the response to the startle pulse, however, in rats treated with PCP a loss of sensorimotor gating leads to a deficit in pre-pulse inhibition such that response to the startle pulse is stronger. This deficit in pre-pulse inhibition of startle is seen in Schizophrenia.

Acclimatized male Sprague-Dawley or Wistar rats that have ad libitum access to food and water at room temperature are divided into five treatment groups:

a) 10 vehicle s.c. and vehicle i.n.
b) 10 PCP s.c. and vehicle i.n.

### c) 10 PCP s.c. and Clozapine i.p.

d) 10 PCP s.c. after 14 days of KLK1 treatment at dose I
e) 10 PCP s.c. after 14 days of KLK1 treatment at dose II
The doses used are: PCP 0.5 to 3.0 mg/kg, Clozapine 1 to 15 mg/kg and KLK1 0.01 to 1000 IU. KLK1 is given one hour before pre-pulse inhibition testing. Clozapine is given ˜30 min before pre-pulse inhibition testing. PCP is given ˜15 minutes before pre-pulse inhibition testing.

Animals are individually placed in a standard startle chamber and allowed to habituate for 2-5 minutes period in the chamber prior to the start of testing. In a pseudorandom manner, each animal is subjected to 10-15 no startle pulse (background noise only) trials, 10-15 startle pulse trials (100-120 dB), 10-15 low pre-pulse (50-90 dB)+startle trials, 10-15 mid pre-pulse (50-90 dB)+startle trials and high pre-pulse (50-90 dB)+startle trials with an inter-trial interval of 10-30s seconds. Movement is measured by the chamber sensors in response to the startle pulse and recorded as the startle amplitude. Pre-pulse inhibition is defined by the percent reduction in startle amplitude in the presence of a pre-pulse compare to the amplitude in the absence of a pre-pulse (1004100×mean of pre-pulse_{(low, mid, or high)}+startle/mean of startle).

After treatment with PCP, the PCP treated-vehicle only group show a statistically significant decrease in percent pre-pulse inhibition compared non-PCP treated group. The clozapine treatment group, as a positive control, shows a statistically significant increase in percentage pre-pulse inhibition compared to the PCP treated-vehicle group. Treatment with KLK1 restores pre-pulse inhibition after PCP treatment, such that there is a statistically significant increase in percent pre-pulse inhibition compared to the PCP treated-vehicle only treatment group.

### Example 2

### PCP Enhanced Immobility Time in a Forced Swim Test Decreased by KLK1 Treatment

Acclimatized Sprague-Dawley rats are divided into control and treatment groups (3 groups). Two treatment groups each receive 10 mg/kg/day PCP injected intraperitoneally for 14 days while the control receives saline. On day 15 one of the treatment groups receives 0.01-1000 IU of KLK1 for 30-60 minutes.

### Forced Swim Test

Rodents become immobile when placed in a water filled glass cylinder without the possibility of escape, which is an unconditioned response to stress (also known as “behavioral despair”). Noda et al. established that repeated PCP injections induce this response (Br. J. Pharmacol., 1995, 116:2531-2537). Therapeutics for treating schizophrenia, specifically negative symptoms, reduced this response.

Rats are subjected to a forced swim test by placement into a Plexiglas@ cylinder (60.5 cm high and 29 cm in diameter) filled with 30 cm of water at 25° C., for six minutes. Immobility time is recorded to determine the mean of the group.

KLK1 is able to decrease the immobility time of mice treated with PCP. As such, the PCP+KLK1 group shows an unexpected and significant decrease in mean immobility time in comparison to the PCP only treatment group.

### Example 3

### KLK1 reduces Sub-Chronic PCP-Induced Cognitive Deficit in the Novel Object Recognition (NOR) Task

The Novel Objection Recognition (NOR) task relies on an animal's natural tendency to explore a novel object instead of a familiar object. Exploration of the novel objects indicates learning and memory (cognition). Less exploration time with a novel object compared to an unaffected control indicates a cognitive impairment.

Acclimatized female Hooded-Lister rats are divided into control and treatment groups. Two treatment groups receive 10 mg/kg/day PCP injected intraperitoneally for 7 days while the control groups receives saline, followed by 7 days without treatment for all groups. On day 15 one of the PCP treatment groups receives 0.01-1000 IU of KLK1 for 30-60 minutes.

Each rat is placed in an open Plexiglas@ box with black walls (52 cm L; 52 cm W; 31 cm H) in which two objects of similar height are placed. Rats are then removed from the box for a short period of time (1 minute) and returned to find that one of the objects has been replaced with a new object. Exploration time of the animal to each of the objects is determined. Exploration time includes the time spent sniffing, licking, touching, standing or sitting on the object.

Prior to this test rats are introduced to the box so that they may become familiar with this environment and this stimulus will therefore not affect test results. Whenever a rat is removed from the box the objects and the box itself are cleaned with 10% alcohol to remove lingering olfactory cues.

All experiments are filmed and analyzed by an experimenter blinded to the treatment groups.

The PCP only treatment group is unable to discriminate between the familiar object and the novel object due to PCP-induced cognitive impairment. Also, there is no statistically significant difference found in mean exploration time between the familiar and novel objects. The PCP+KLK1 treatment group shows that KLK1 is able to inhibit PCP-induced cognitive impairment due the observation of an unexpected and significant increase in mean exploration time towards the novel object compared to the familiar object.

This result indicates KLK1 is able to reduce cognitive deficit-like symptoms thought to replicate cognitive deficits of psychiatric disorders.

### Example 4

### PCP-Induced Neurodegeneration via GSK-3β Activation is Prevented with KLK1 Treatment

Sprague-Dawley rat embryonic forebrain cells (E18-E19) are dissociated using cold Hanks balanced salt solution (HBSS) without Mg²⁺ or Ca²⁺, and re-plated in polylysine (5 mg/ml) coated multi-well plates at 10×10⁵ cell/ml and grown in Neurobasal Medium (Invitrogen, Carlsbad, Calif.) supplemented with 0.05 mM L-glutamine and 10% B27 (Invitrogen) at 37° C. in 5% CO₂. The culture media is replaced every four days.

After 14 days, cells are treated with 1 μM PCP over various time frames: 0, 1, 3, 6, 12, 24 and 48 hours. After the end of the specific time points the cells are washed with PBS, and lysed with RIPA buffer (Pierce Biotechnology, Rockford, Ill.). The cell extracts are centrifuged at 20 000×g, the supernatant is collected, and protein concentration is determined using the BCA Protein Assay Kit (Pierce). Using 30 μg of protein cell extracts, each time point is Western blotted to determine the presence and for quantification of phosphorylated GSK-3β^{S9}(inactive) and activated caspase-3 (17 kDa). The relative band density of each is compared to the control (non-PCP treated cell lysate) at each time point and plotted on a density graph as a percentage of the control. The graph shows that over time in PCP only treated cells, the level of phosphorylated GSK-3β^{S9} decreases while the level of activated capase-3 (17 kDa) increases up to the 24 hour time point. This shows that PCP treatment leads to GSK-3β activation, which results in activation of casapse-3 downstream by active GSK-3β. This trend is indicative of neuronal cell death via caspase-3 activation, which is a hallmark of the neurodegeneration observed in psychiatric disorders.

This same procedure is repeated with cells treated with PCP (1 μM) and KLK1 (within a dose range of 0.001 to 1000 IU) over various time frames: 0, 1, 3, 6, 12, 24 and 48 hours. The resulting graph shows that the addition of KLK1 prevents PCP induced neurodegeneration. The trend of decreased phosphorylation of GSK-3β^{S9} and increased active caspase-3 over time is attenuated by KLK1.

### Example 5

### Prevention of Pre-Pulse Inhibition of Startle Deficit after Treatment with KLK1 in Mice

Acclimatized male mice that have ad libitum access to food and water at room temperature are divided into five treatment groups:

a) 10 vehicle s.c. and vehicle i.n.
b) 10 PCP s.c. and vehicle i.n.

### c) 10 PCP s.c. and Clozapine i.p.

d) 10 PCP s.c. after 14 days of KLK1 treatment at dose I
e) 10 PCP s.c. after 14 days of KLK1 treatment at dose II

The doses used are: PCP 1.0 to 10.0 mg/kg, Clozapine 1 to 15 mg/kg and KLK1 0.01 to 1000 IU. KLK1 is given one hour before pre-pulse inhibition testing. Clozapine is given ˜30 min before pre-pulse inhibition testing. PCP is given ˜15 minutes before pre-pulse inhibition testing.

Animals are individually placed in a standard startle chamber and allowed to habituate for 2-5 minutes period in the chamber prior to the start of testing. In a pseudorandom manner, each animal is subjected to 10-15 no startle pulse (background noise only) trials, 10-15 startle pulse trials (100-120 dB), 10-15 low pre-pulse (50-90 dB)+startle trials, 10-15 mid pre-pulse (50-90 dB)+startle trials and high pre-pulse (50-90 dB)+startle trials with an inter-trial interval of 10-30s seconds. Movement is measured by the chamber sensors in response to the startle pulse and recorded as the startle amplitude. Pre-pulse inhibition is defined by the percent reduction in startle amplitude in the presence of a pre-pulse compare to the amplitude in the absence of a pre-pulse (100-(100×mean of pre-pulse_{(low, mid, or high)}+startle/mean of startle).

After treatment with PCP, the PCP treated-vehicle only group show a statistically significant decrease in percent pre-pulse inhibition compared non-PCP treated group. The clozapine treatment group, as a positive control, shows a statistically significant increase in percentage pre-pulse inhibition compared to the PCP treated-vehicle group. Treatment with KLK1 restores pre-pulse inhibition after PCP treatment, such that there is a statistically significant increase in percent pre-pulse inhibition compared to the PCP treated-vehicle only treatment group.

1. A method of treating a psychiatric disorder comprising administering tissue kallikrein, fragment, or variant thereof; wherein the psychiatric disorder has a dysfunctional PI3K/Akt/GSK-3β pathway.

2. The method of claim 1, wherein the psychiatric disorder is schizophrenia.

3. The method of claim 1, wherein the psychiatric disorder is bipolar disorder.

4. The method of claim 1, wherein the psychiatric disorder is schizophrenic spectrum.

5. The method of claim 1 wherein the tissue kallikrein, fragment, or variant thereof comprises an amino acid sequence selected from the group consisting of SEQ ID NO:1 and SEQ ID NO:2.

6. The method of claim 1 wherein the tissue kallikrein, fragment, or variant thereof comprises an amino acid sequence 80% identical to a sequence selected from the group consisting of SEQ ID NO:1 and SEQ ID NO:2.

7. The method of claim 1 wherein the administering comprises administration of 1 to 1000 International Units per day, administered orally.

8. The method of claim 1 wherein the administering comprises administration of 1 to 5000 International Units per day, administered intranasally.

9. The method of claim 1 wherein the administration is in combination with a conventional psychiatric disorder drug.

10. The method of claim 9 wherein the conventional psychiatric disorder drug is selected from the group consisting of trifluoperazine, fluanxol. Loxapac, loxitane, etrafon, trilafon, thorazine, halopoidol, fluphenazine, decanoate, aripiprazole, clozapine, ziprasidone, resperidone, questiapire, olanzapine, iloperidone, DTA 20 IA, DV 127090, ORG 5222, osanetant, MEM 3454, aripiprazole, asenapine, carbamezapine, fluoxetine, alanzapine, lamotrigine, lithium, loanzapine, oxcarbazepine, quetiapine, risperidone, topirimate, valproic acid, divalproex sodium, and ziprasidone.

11-15. (canceled)
